(19) United States
(12) Patent Application Publication
(10) Pub. No.: US 2003/0059458 A1
(43) Pub. Date: Mar. 27, 2003

(54) Encapsulated carob fibers having improved product properties, process for production thereof and use thereof

(76) Inventors: Bernd Haber, Mainz (DE); Hans-Ulrich Ter Meer, Frankfurt (DE)

Correspondence Address: ProPat, L.L.C., 2912 Crosby Road, Charlotte, NC, 28211-2815, US

(21) Appl. No.: 10/242,057

(22) Filed: Sep. 12, 2002

(30) Foreign Application Priority Data
Sep. 25, 2001 (DE) 101 47 156.4

(51) Int. Cl. A61K 47/00; A61K 35/78

(52) U.S. Cl. 424/439; 424/757

(57) ABSTRACT

The invention relates to encapsulated dietary fiber consisting of a core of essentially water-insoluble carob fiber and an encapsulating material of at least one water-soluble dietary fiber. The invention also relates to a process for producing such encapsulations and their use, in particular in foods, animal feed, cosmetics and pharmaceuticals.

The invention relates to encapsulated carob fibers consisting of insoluble carob fibers and one or more encapsulating materials. In addition, the invention relates not only to the production process but also to the use of these stable encapsulated carob fibers.

### BACKGROUND OF THE INVENTION

Dietary fiber is plant food components which cannot be digested by humans. They comprise indigestible polysaccharides, oligosaccharides, lignin and associated substances. Dietary fiber is highly important from the nutritional aspect and, in addition to an effect promoting digestion can also have a regulating action on blood cholesterol and the blood glucose level. Furthermore, in the large intestine, fermentable oligosaccharides and polysaccharides display a prebiotic activity. A high-fiber diet is also reported as a cause for the relatively rare occurrence of bowel cancer in Africa or in certain population groups. In most industrial countries, however, the consumption of dietary fiber is considerably below the daily dose of at least 30 g which is recommended by nutritionists. An increased supply of dietary fiber is therefore desirable from the nutritional aspect. This includes achieving a balanced ratio between soluble and insoluble dietary fiber in the diet.

Since this knowledge is becoming increasingly established, the desire on the part of the consumer for foods having a specific health benefit is becoming increasingly strong. In practice, when foods are enriched with dietary fiber, there is the problem that texture and mouth feel and also color can suffer.

Carob is a Mediterranean fruit which is rich is carbohydrates, dietary fiber and polyphenols. Compared with customary sources of dietary fiber such as cereals and fruits and vegetables, the insoluble dietary fiber produced from the fruit flesh of carob, termed in practice carob fiber, has a unique composition and, in addition to its action promoting digestion, has additional health benefits (Pérez-Olleros et al., 1999, J. Sci. Food Agric. 79, 173-178). EP-B-0 616 780 describes a production process for high-fiber compositions from carob. The special production process produces compositions which are rich in insoluble carob fibers.

Because of its various bioactive constituents, carob fiber is an ingredient of interest for the functional enrichment of a broad variety of foods. Owing to its insolubility and dark color, the use of carob fiber, however, is restricted to certain food groups. The dark color is more noticeable in foods which are naturally lighter. In addition, when carob fiber is added, there is frequently a marked sensory perception of the particles in the food matrix (“irritating off-taste”).

The technique of microencapsulation is used in various sectors (for example colloid and polymer chemistry, suspension and drying technology, pharmaceutical industry, food industry). Depending on the field of application, the primary role is protecting the enveloped material from chemical and/or physical changes or the controlled release of substances. However, in addition, odor, taste or an adverse mouth feel of the encapsulated substance can also be masked. For sometime microencapsulation has also increasingly been used in food technology. The spray-drying process is one of the most frequently used processes for encapsulating various substances in the food industry. However, other coating processes, such as the fluidized bed process or the Wurster process are also suitable for food ingredients such as flavors, spices, fats and fatty acids, vitamins, colors etc.

U.S. Pat. No. 4,619,831 describes dietary fiber compositions, and a production process therefor, which compositions are formed from insoluble and soluble dietary fiber. In this process the insoluble dietary fiber is coated with a thin film of soluble dietary fiber. This is achieved by simple mixing or a coating process.

Mixtures of soluble and insoluble dietary fiber are also described in EP-A-0 756 828. The combination serves to optimize the nutritional benefit of dietary fiber.

Zein-encapsulated dietary fiber particles, in particular guar gum, are described as constituents of cholesterol-lowering foods in U.S. Pat. No. 5,545,414. Guar gum is there enclosed by a Zein encapsulation.

U.S. Pat. No. 5,599,556 describes prolamine coatings to mask unwanted flavor impressions. Nutritional substances such as dietary fiber are also mentioned there.

A technology is described for fiber which is “invisible” to water, in which dietary fiber, by means of encapsulation with milk proteins, is made available for a broader use in foods (“New Technology Boosts Fiber in Foods”, C. I. Onwulata, Agricultural Research, March 2001, 14-15).

The previously described compositions of soluble and insoluble dietary fiber are frequently pure mixed compositions without a particular structure or, because of inadequate stabilities, do not represent a satisfactory solution for broader use in foods. In particular they would not be suitable for making available for broad use in sensitive foods dietary fibers such as carob fiber which have a long term stable dark color. From a nutritional aspect, it would be particularly desirable to stabilize, with soluble dietary fiber, a dietary fiber composition of insoluble dietary fiber such as carob fiber. Combining the different actions of soluble and insoluble dietary fiber, in the form of encapsulated dietary fiber, in addition to improving the applicability of the insoluble dietary fiber, achieves a particularly good ingredient for supplementing foods with dietary fiber in a nutritionally balanced manner.

It was therefore an object of the present invention to provide a dietary fiber composition of carob fiber which is of particular interest nutritionally which at the same time

1. Is lighter in color than carob fiber itself and as a result reduces coloring effects of carob fiber on foods during processing and storage,

2. At the same time gives sufficient stability for a broad food use,

3. Minimizes adverse sensory effects of carob fiber on the texture of food and

4. For stabilization where possible uses soluble dietary fiber and thus represents a food ingredient of particular interest nutritionally.

### BRIEF DESCRIPTION OF THE INVENTION

This object is achieved by an encapsulated dietary fiber, the core of which comprises carob fiber, and the encapsulating material of which consists of at least one soluble dietary fiber and if appropriate other stabilizing materials. Since, hitherto, to enrich foods the dietary fiber has usually been added individually or previous enveloping solutions still did not display sufficient stabilities in foods, the universal stabilization method described below and the resultant substances are a considerable improvement compared with the prior art. This applies in particular to foods which are sensitive on account of their color and texture, in which carob fiber may be used only with difficulty.

### DETAILED DESCRIPTION OF THE INVENTION

In addition the present invention provides dietary fiber compositions in which various effects of soluble and insoluble dietary fiber are ideally combined. As a result ideal food ingredients may be generated which can be used optimally for enriching foods with dietary fiber or as functional ingredients.

The inventive carob compositions serve as an ingredient of foods, animal feed or pharmaceuticals. They can serve for pure dietary fiber enrichment and/or they can be used for the targeted supply of functional ingredients having a particular additional benefit. In addition the inventive dietary fiber compositions are constituents which are added in a targeted manner to products which supplement the human and/or animal diet (food supplements, dietary supplements).

The carob fibers used in the inventive compositions can be produced by known processes from carob fruit. A process which is suitable in particular is the process claimed in EP-A-0 616 780. The carob fibers thus produced are substantially water-insoluble, that is to say they still comprise a maximum of 5 to 25% by weight, preferably 10-15% by weight, of water-soluble constituents.

In the inventive compositions, the insoluble carob fibers are surrounded on all sides by an envelope. Suitable enveloping substances are, in particular, soluble dietary fibers, which are listed in Table 1. They surprisingly improve the color of the carob fibers and at the same time improve the taste and odor properties. In addition to naturally occurring soluble dietary fiber, polysaccharides prepared by fermentation or chemically modified polysaccharides as listed in Table 1 are also suitable. Auxiliary materials can be used to increase the stability of the compositions. Suitable materials for this are, for example, emulsifiers, peptides or proteins, as also listed in Table 1.

In the inventive compositions, the nutritionally desirable effects of the dietary fiber, for example on intestinal peristalsis, on blood cholesterol values or on postprandial glucose values, are retained.

TABLE 1
Examples of suitable encapsulating materials
in the context of the invention

| Soluble dietary | Polysaccharides | Chemically | |
|---|---|---|---|
| fiber, natural | produced by | modified | Auxiliary |
| origin | fermentation | polysaccharides | materials |
| Gum arabic | Dextrans | Carboxymethyl | Lecithin |
| Tragacanth | Xanthan | cellulose | Tween |
| Karaya gum | Curdlan | Methyl cellulose | Albumins |
| Carob bean meal | | Hydroxypropyl | Gelatin |
| Guar gum | | cellulose | Caseins |
| Alginates | | Hydroxypropyl | Minerals |
| Carrageenans | | methyl | Acidulants |
| Agar | | cellulose | Sweeteners |
| Psyllium | | Low-esterified | Flavors |
| β-glucans | | pectins | Protein partial |
| Inulin | | Propylene glycol | hydrolyzates |
| Oligosaccharides | | alginates | Plant proteins |
| Maltodextrins | | Methyl | Preservatives |
| Pectins | | hydroxyethyl | Antioxidants |
| Fenugreek fiber | | cellulose | Pigments |
| Arabinogalactans | | Ethyl | Dyes |
| Galactomannans | | hydroxyethyl | |
| Arabinoxylans | | cellulose | |
| | | Hydrolyzed guar | |
| | | gum | |
| | | Chemically | |
| | | modified starches | |

The encapsulated carob fibers are spherical or polygonal structures having, in the unprocessed state, a mean diameter of 1 μm to 200 μm, preferably 10 to 100 μm, in particular <70 μm. In the processed state, that is to say in food, the particle diameter is unchanged, but it can also increase up to 5 fold.

The content of insoluble carob fibers, depending on the desired effect in the product in which the fiber composition is to be used, is 10 to 90% by weight, preferably 20 to 50% by weight, in particular around 33% by weight. The content of soluble, enveloping fiber can, depending on the field of use, be from 10 to 90% by weight, preferably 20 to 75% by weight, in particular around 33% by weight. The addition of auxiliary materials depends on the stability to be achieved and the effect desired and can be 0-60% by weight, but preferably less than 33% by weight. The abovementioned percentages by weight are based on the encapsulated carob fiber.

To produce the inventive fiber composition, expediently a procedure is followed such that the insoluble carob fiber and the soluble dietary fiber and if appropriate the auxiliary materials are introduced into a liquid medium, usually a solvent or dispersant. The resultant suspension is then mixed homogeneously and then freed from solvent or dispersant. Also, a solution of one or more soluble dietary fibers and if appropriate one or more auxiliary materials can first be introduced and the insoluble carob fibers homogeneously suspended therein. In this case also the solvent or dispersant is then taken off.

A liquid medium which has proved to be very highly suitable is water. In addition, it is expedient also to add auxiliary materials which contribute to the envelope formation.

The solvent or dispersant is removed by known drying processes, for example spray-drying, fluid-bed drying, freeze-drying inter alia, but preferably by spray-drying. In this process, usually, a one-component nozzle is used for atomizing which ensures the formation of sufficiently small particles during the spraying operation. Preferably, nozzles having a nozzle diameter of 0.1 to 2.0 mm are used. However, it can also prove to be expedient that the envelope material is not combined with the mixture until immediately during encapsulation, so that the materials are combined in the dryer via a two-component nozzle.

Surprisingly, the encapsulated carob fibers thus produced have markedly reduced coloration compared with nonencapsulated fibers. In addition, the unwanted adverse perceptions of the insoluble carob fibers, with respect to the sensory and technological aspects, are markedly improved. Furthermore, it has been found that these dietary fiber compositions are distinguished after the drying process by a high stability and can therefore be used in sensitive foods in which the non-encapsulated carob fibers cannot be used without adverse effect on color and texture.

The invention can be used in very many food groups, such as milk products (yogurts, quark cheeses, fermented milk products, fresh cheese, cheese preparations), bakery products (bread, rolls, patisserie products), beverages, also in food supplements, in animal nutrition (pets and small animals; farm animals) and in cosmetics and in pharmaceuticals, etc.

The invention is described below with reference to examples.

### EXAMPLE 1

### Production of Encapsulated Carob Fiber

1% by weight of insoluble carob fiber (Caromax™, Nutrinova, Frankfurt, Germany) is suspended in water. The encapsulated material consisting of 1% by weight soluble dietary fiber gum arabic and 1% by weight of gelatin is also suspended in water. Mixing the two suspensions produces a sprayable dispersion. This dispersion, to ensure uniform distribution of the dispersants, is continuously stirred at 500 rpm during the subsequent spray-drying. The drying process was carried out using the following parameter settings:

Drying air temperature: 170 to 185° C.

Exhaust temperature: 55 to 60° C.

Spraying pressure: 1 bar

Inlet pressure: 0.01 bar

The resultant beige-white powder is very fine and of a free-flowing particulate character.

### EXAMPLE 2

### Morphological Change on Encapsulation

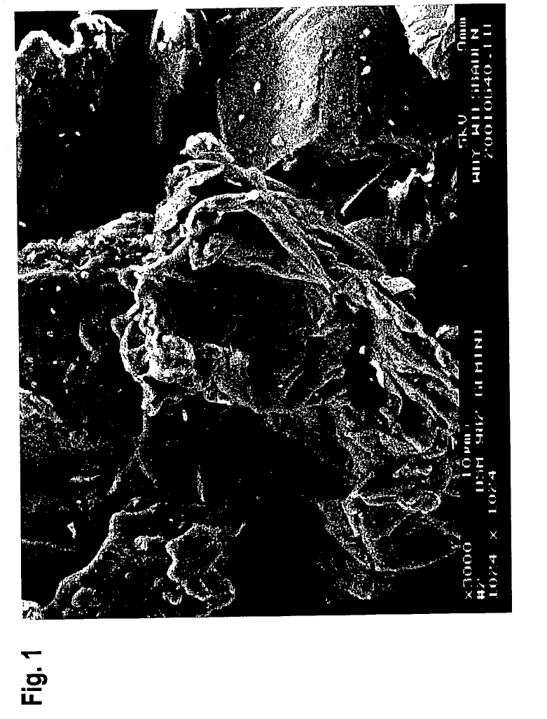

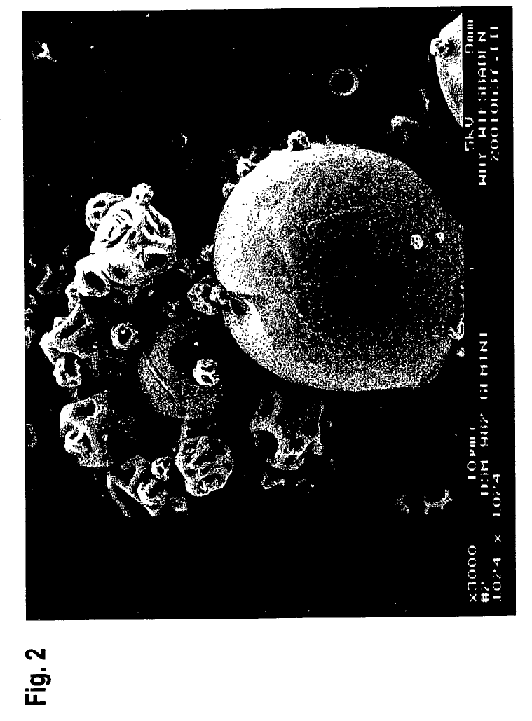

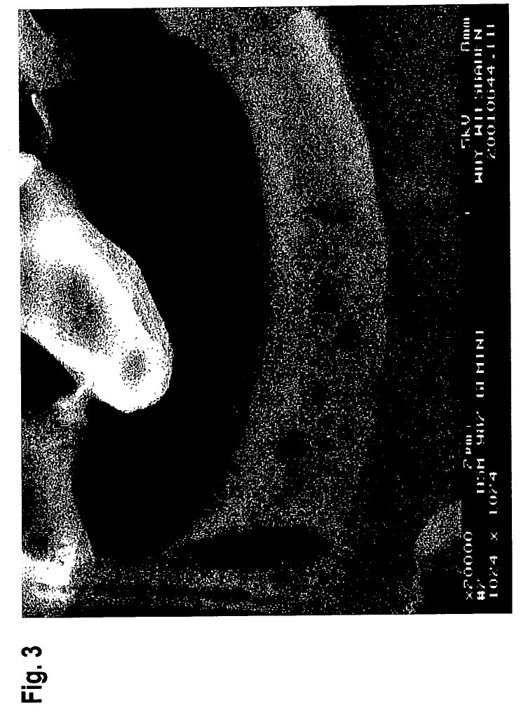

The encapsulated carob fibers produced as in Example 1 were studied by electron microscope. It was found that the originally irregularly shaped particulate carob fibers (FIG. 1) were changed by the encapsulation process into uniformly rounded particles. (FIG. 2). In addition, very small (diameter approximately 1 to 2 μm) particles are formed from pure encapsulating material. In the sectioning method, it is found that the carob fibers according to the process described in Example 1 have an encapsulating thickness of 1 to 2 μm (FIG. 3).

### EXAMPLE 3

### Production of Encapsulated Carob Fiber

9.375 g of gelatin were stirred into 500 g of water and dissolved with heating. After dissolution and cooling to approximately 30° C., under further stirring, 9.375 g of methyl hydroxyethyl cellulose (MHEC: ®Tylopur MH50G4, Clariant, Wiesbaden, Germany) were added and dissolved with further stirring. 6.25 g of carob fiber (Caromax™) were dispersed in this solution. This solution, to ensure uniform distribution of dispersants, was continuously stirred at 500 rpm during the subsequent spray-drying.

The drying process was carried out using the following parameter settings:

Drying air temperature: 160° C.

Exhaust temperature: 75° C.

Spraying pressure: 1 bar

Inlet pressure: 0.01 bar

The resultant whitish, only slightly beige powder is very fine and is markedly lighter in color than the powder described in Example 1.

### EXAMPLE 4

### Production of a High-fiber Yogurt

9 g of the encapsulated carob fibers as described in Example 1 were stirred into 200 g of a natural yogurt. This gave an enrichment in the end product of 3% by weight of dietary fiber. The color of the yogurt was changed only insignificantly by the addition. The thus enriched natural yogurt was further distinguished by a markedly creamier, more full-bodied mouthfeel compared with the non-enriched control. The stability of the encapsulated fibers in this yogurt was followed for a period of more than 14 days and during the entire time no taste or visual changes were found.

### EXAMPLE 5

### Use in Bakery Products (Muffin)

TABLE 2
Ingredients for muffin production

| | Formulation using | Reference formula |
|---|---|---|
| | encapsulated carob | using carob fibers |
| | fibers | (nonencapsulated) |
| Ingredients | [g] | [g] |
| Flour, type 405 | 48.65 | 54.1 |
| Baking powder | 1.75 | 1.75 |
| Soda | 0.75 | 0.75 |
| Whole egg | 22.5 | 22.5 |
| Sugar | 35 | 35 |
| Margarine | 31.25 | 31.25 |
| Carob fibers, | 16.45 | — |
| encapsulated | | |
| Carob fibers, | — | 10.9 |
| nonencapsulated | | |
| Buttermilk | 62.5 | 62.5 |
| Σ | 218.75 | 218.75 |
| Initial dough weight | 60 g per molded piece | 60 g per molded piece |
| Muffin product | 50.2; 50.4; 50.2 | 50.4; 50.4; 50.3 |
| weight | ø50.3 | ø50.4 |
| Baking loss % | 16.17% | 16.0% |
| Dietary fiber | 5.98% | 5.93% |
| content % | | |

Processing: mix flour, (encapsulated) carob fiber as described in Example 1, baking powder and soda. Agitate sugar, margarine and whole eggs to a foam, add the buttermilk and the dry ingredients and briefly stir. Then pour into muffin molds and bake at 160° C. in a forced air circulation oven for 25 min.

Sensory comparison of the muffins made according to the two formulas found (Table 3) that the muffins baked using encapsulated carob fiber were rated significantly better than those baked with pure carob fiber.

TABLE 3
Sensory rating of bakery products

| | Formula using | Reference formula |
|---|---|---|
| | encapsulated carob | using carob fibers |
| Evaluation: | fibers | (nonencapsulated) |
| Appearance/color | | |
| external: | light brown | chocolate brown |
| internal: | beige-brown | chocolate brown |
| | fine porosity, | fine porosity, |
| | similar to | similar to |
| | sponge cake | sponge cake |
| Odor | typical of egg, | markedly of carob, |
| | slightly of carob | malty, burnt |
| Taste | initially neutral, | markedly of carob, |
| | later slightly of | malty |
| | carob, slightly | |
| | malty; | |
| Texture | very light, moist, | very light, moist, |
| | particles scarcely | particles perceptible |
| | perceptible | |
| Overall | good | acceptable |
| impression | | |

1. An encapsulated dietary fiber consisting of a core of essentially water-insoluble carob fiber and an encapsulating material of at least one water-soluble dietary fiber.

2. An encapsulated dietary fiber as claimed in claim 1, wherein the encapsulating material further comprises at least one auxiliary material.

3. An encapsulated dietary fiber as claimed in claim 1, wherein the water-soluble dietary fiber is selected from one or more of the groups of substances comprising: water-soluble dietary fibers of natural origin, polysaccharides produced by fermentation and chemically modified polysaccharides.

4. An encapsulated dietary fiber as claimed in claim 3, wherein the water-soluble dietary fiber of natural origin is selected from one or more of the substances consisting of: gum arabic, tragacanth, karaya gum, carob bean meal, guar gum, alginates, carrageenans, agar, psyllium, β-glucans, inulin, oligosaccharides, maltodextrins, pectins, fenugreek fiber, arabinogalactans, galactomannans and arabinoxylans.

5. An encapsulated dietary fiber as claimed in claim 3, wherein the polysaccharide produced by fermentation is selected from one or more of the substances comprising: dextrans, xanthan and curdlan.

6. An encapsulated dietary fiber as claimed in claim 3, wherein the chemically modified polysaccharide is selected from one or more of the substances comprising: carboxymethyl cellulose, methyl cellulose, hydroxypropyl cellulose, hydroxypropyl methyl cellulose, low-esterified pectins, propylene glycol alginates, methyl hydroxyethyl cellulose, ethyl hydroxyethyl cellulose, hydrolyzed guar gum and chemically modified starches.

7. An encapsulated dietary fiber as claimed in claim 2, wherein the auxiliary material is selected from one or more of the substances comprising: lecithin, Tween, albumins, gelatin, caseins, minerals, acidulants, sweeteners, flavors, protein partial hydrolyzates, plant proteins, preservatives, antioxidants, pigments and dyes.

8. An encapsulated dietary fiber as claimed in claim 1, wherein the carob fiber is present in the encapsulated fiber in an amount of about 10 to about 90% by weight (based on the encapsulated fiber).

9. An encapsulated dietary fiber as claimed in claim 1, wherein the water-soluble dietary fiber is present in the encapsulated fiber in an amount of about 10 to about 90% by weight (based on the encapsulated fiber).

10. An encapsulated dietary fiber as claimed in claim 2, wherein the auxiliary material is present in the encapsulated fiber in an amount of >0 to about 60% by weight (based on the encapsulated fiber).

11. An encapsulated dietary fiber as claimed in claim 1, wherein it has a spherical or polygonal shape.

12. An encapsulated dietary fiber as claimed in claim 11, wherein it has, in the unprocessed state, a mean diameter of about 1 to about 200 μm.

13. A process for producing an encapsulated dietary fiber as claimed in claim 1, which comprises suspending the carob fiber and the water-soluble dietary fiber in a liquid medium and then completely or partially removing the liquid medium.

14. A process as claimed in claim 13, wherein the liquid medium is removed by spray-drying.

15. The method of making a food or food supplement comprising the step of incorporating an encapsulated dietary fiber as claimed in claim 1 in a food or food supplement.

16. A method of making an animal nutrition comprising the step of incorporating an encapsulated dietary fiber as claimed in claim 1 in an animal nutrition.

17. A method of making a pharmaceutical comprising the step of incorporating an encapsulated dietary fiber as claimed in claim 1 in a pharmaceutical.

18. A method of making a cosmetic comprising the step of incorporating an encapsulated dietary fiber as claimed in claim 1 in a cosmetic.
